(19) United States
(12) Patent Application Publication
(10) Pub. No.: US 2016/0347836 A1
(43) Pub. Date: Dec. 1, 2016

(54) TREATMENT OF HODGKIN'S LYMPHOMA USING AN ANTI-PD-1 ANTIBODY

(71) Applicant: BRISTOL-MYERS SQUIBB COMPANY, Princeton, NJ (US)

(72) Inventor: Joseph Grosso, New York, NY (US)

(21) Appl. No.: 15/164,960

(22) Filed: May 26, 2016

Related U.S. Application Data
(60) Provisional application No. 62/167,715, filed on May 28, 2015.

(51) Int. Cl. C07K 16/28; C12Q 1/68; G01N 33/574; A61K 39/395; A61K 45/06

(52) U.S. Cl. CPC C07K 16/2803; A61K 39/3955; A61K 45/06; G01N 33/57407; C12Q 1/6886; C12Q 2600/158; G01N 2333/4704; A61K 2039/505

(57) ABSTRACT

This disclosure provides methods for treating Hodgkin's lymphoma in a subject comprising administering to the subject an anti-Programmed Death-1 (PD-1) antibody. In some embodiments, this invention relates to methods for treating Hodgkin's lymphoma in a subject comprising administering to the subject a combination of an anti-cancer agent which is an anti-Programmed Death-1 (PD-1) antibody and another anti-cancer agent such as an anti-Cytotoxic T-Lymphocyte Antigen-4 (CTLA-4) antibody.

### CROSS-REFERENCE TO RELATED APPLICATIONS

This application claims priority to U.S. Provisional Application Ser. No. 62/167,715, filed May 28, 2015, the entire content of which is incorporated herein by reference.

Throughout this application, various publications are referenced in parentheses by author name and date, or by patent No. or patent Publication No. Full citations for these publications can be found at the end of the specification immediately preceding the claims. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

### FIELD OF THE INVENTION

This invention relates to methods for treating Hodgkin's lymphoma in a subject comprising administering to the subject an anti-Programmed Death-1 (PD-1) antibody. In some embodiments, this invention relates to methods for treating Hodgkin's lymphoma in a subject comprising administering to the subject a combination of an anti-cancer agent which is an anti-Programmed Death-1 (PD-1) antibody and another anti-cancer agent such as an anti-Cytotoxic T-Lymphocyte Antigen-4 (CTLA-4) antibody. This invention also relates to methods of identifying a subject with a Hodgkin's lymphoma who is suitable for immunotherapy treatment by measuring genetic alterations of the PD-1 ligand loci and associated protein expression.

### BACKGROUND OF THE INVENTION

Human cancers harbor numerous genetic and epigenetic alterations, generating neoantigens potentially recognizable by the immune system (Sjoblom et al., 2006). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

Until recently, cancer immunotherapy had focused substantial effort on approaches that enhance anti-tumor immune responses by adoptive-transfer of activated effector cells, immunization against relevant antigens, or providing non-specific immune-stimulatory agents such as cytokines. In the past decade, however, intensive efforts to develop specific immune checkpoint pathway inhibitors have begun to provide new immunotherapeutic approaches for treating cancer, including the development of an antibody (Ab), ipilimumab (YERVOY®), that binds to and inhibits CTLA-4 for the treatment of patients with advanced melanoma (Hodi et al., 2010) and the development of Abs such as nivolumab and pembrolizumab (formerly lambrolizumab; USAN Council Statement, 2013) that bind specifically to the Programmed Death-1 (PD-1) receptor and block the inhibitory PD-1/PD-1 ligand pathway (Topalian et al., 2012a, b; Topalian et al., 2014; Hamid et al., 2013; Hamid and Carvajal, 2013; McDermott and Atkins, 2013).

The PD-1 pathway serves as an immune checkpoint to limit T-cell-mediated immune responses.⁶ Sustained PD-1 expression and signaling results in T-cell “exhaustion,” a temporary inhibition of activation and proliferation that can be reversed upon removal of the PD-1 signal. Furthermore, PD-L1 also promotes the induction and maintenance of PD-1+ T-regulatory cells.⁷ By expressing PD-1 ligands on the cell surface and engaging PD-1 on immune effector cells, tumors can co-opt this pathway in order to evade the immune response.⁸

Recently, PD-1-blocking antibodies have been used to enhance immune responses against solid tumors, resulting in clinical responses that can be durable and achieved with an acceptable safety profile.⁹⁻¹³ At least in some solid tumors, PD-L1 expression appears to be the best predictor of response to PD-1-blocking antibodies,^{14, 15} although the basis for PD-1 ligand overexpression in these diseases remains undefined. There are also preliminary data supporting empiric PD-1 blockade as a therapeutic strategy in hematologic malignancies.^{14, 16-21}

In order to survive in an immunocompetent host, human tumors must evolve mechanisms to disable immune responses against tumor neoantigens. Classical Hodgkin lymphoma (cHL) is characterized pathologically by small numbers of malignant Reed-Sternberg (RS) cells within an extensive inflammatory infiltrate.^{1, 2} However, there is little evidence of an effective antitumor immune response, suggesting that immune evasion pathways are central to the tumor's survival in the host. Indeed, the Hodgkin RS (HRS) cells express molecules that limit the efficacy of T-cell responses.¹⁻³

Recent analyses integrating high-resolution copy-number data and transcriptional profiles identified the immune checkpoint programmed death-1 (PD-1) ligands, PD-L1 and PD-L2, as key targets of chromosome 9p24.1 amplification, a recurrent genetic abnormality in cHL.¹ The 9p24.1 amplicon also includes JAK2, and gene dosage-dependent JAK-STAT activity further induces PD-1 ligand transcription.¹ These copy-number-dependent mechanisms, as well as other less frequent rearrangements,⁴ lead to genetically determined overexpression of the PD-1 ligands on the HRS cell surface. Epstein-Barr virus (EBV) infection, also common in cHL, is an additional mechanism of PD-L1 overexpression, consistent with the virus's known ability to usurp the PD-1 pathway to allow viral persistence in the host.⁵ As a result of the two complementary pathways of 9p24.1 alterations and EBV infection, over 80% of cHL cases have increased surface expression of PD-L1,⁶ suggesting a central role for the PD-1 pathway in this disease.

Classic Hodgkin's lymphomas include small numbers of malignant Reed-Sternberg cells within an extensive but ineffective inflammatory and immune-cell infiltrate._{14, 15} The genes encoding the PD-1 ligands, PDL1 and PDL2 (also called CD274 and PDCDILG2, respectively), are key targets of chromosome 9p24.1 amplification, a recurrent genetic abnormality in the nodular-sclerosis type of Hodgkin's lymphoma.14 The 9p24.1 amplicon also includes JAK2, and gene dose-dependent JAK-STAT activity further induces PD-1 ligand transcription.₁₄ These copy-number-dependent mechanisms and less frequent chromosomal rearrangements lead to overexpression of the PD-1 ligands on Reed-Sternberg cells in patients with Hodgkin's lymphoma. Epstein-Barr virus (EBV) infection also increases the expression of PD-1 ligands in EBV-positive Hodgkin's lymphomas.₁₇

The complementary mechanisms of PD-1 ligand overexpression in Hodgkin's lymphoma suggest that this disease may have genetically determined vulnerability to PD-1 blockade. Coamplification of PDL1 and PDL2 on chromosome 9p24.1 suggests receptor rather than selective ligand blockade as a treatment strategy. For these reasons, Hodgkin's lymphoma was included as a cohort-expansion group in a phase 1 study of nivolumab, a fully human monoclonal IgG4 antibody directed against PD-1, in patients with relapsed or refractory hematologic cancer.

### SUMMARY OF THE INVENTION

The present invention relates to a method for treating a subject afflicted with a Hodgkin's lymphoma comprising administering to the subject a therapeutically effective amount of an antibody or an antigen-binding portion thereof that binds specifically to a Programmed Death-1 (PD-1) receptor and inhibits PD-1 activity (“anti-PD-1 antibody”).

In other embodiments, the antibody or an antigen-binding portion thereof cross-competes with nivolumab for binding to human PD-1. In some embodiments, the antibody or an antigen-binding portion thereof is a chimeric, humanized or human monoclonal anti-PD-1 antibody or an antigen-binding portion thereof. In one embodiment, the anti-PD-1 antibody is nivolumab. In another embodiment, the anti-PD-1 antibody is pembrolizumab.

In some embodiments, the subject is further treated with an anti-cancer therapy concurrently with, prior to, or after the administering. In some embodiments, the anti-cancer therapy comprises administering an anti-cancer agent. In one embodiment, the anti-cancer therapy comprises a radiation therapy or chemotherapy.

In some embodiments, the anti-cancer agent comprises a second antibody or an antigen-binding portion thereof. In one embodiment, the second antibody or an antigen-binding portion thereof binds specifically to Cytotoxic T-Lymphocyte Antigen-4 (CTLA-4) and inhibits CTLA-4 activity (“anti-CTLA-4 antibody”). In another embodiment, the anti-CTLA-4 antibody or antigen-binding portion thereof cross-competes with ipilimumab for binding to human CTLA-4. In other embodiments, the anti-CTLA-4 antibody or antigen-binding portion thereof is a chimeric, humanized or human monoclonal antibody or an antigen-binding portion thereof. In one embodiment, the anti-CTLA-4 antibody is ipilimumab.

In some embodiments, the anti-PD-1 antibody or an antigen-binding portion thereof is administered as monotherapy at a dose of about 3 mg/kg body weight. In other embodiments, the anti-PD-1 antibody or an antigen-binding portion thereof is administered at a dose of about 3 mg/kg body weight in combination with an anti-CTLA-4 antibody or an antigen-binding portion thereof, which is administered at a dose of about 1 mg/kg body weight.

In certain embodiments, the present invention is directed to any method disclosed herein comprising: 1) an induction phase, wherein the anti-PD-1 or antigen-binding portions thereof is administered in combination in 2, 4, 6, 8 or 10 doses, each dose ranging from about 0.1 mg/kg to about 10.0 mg/kg body weight administered at least once every 2, 3, or 4 weeks; 2) followed by a maintenance phase, wherein the anti-PD-1 antibody or antigen-binding portion thereof is repeatedly administered at a dose ranging from about 0.1 mg/kg to about 10 mg/kg at least once every 2, 3 or 4 weeks.

Other features and advantages of the instant invention will be apparent from the following detailed description and examples which should not be construed as limiting. The contents of all cited references, including scientific articles, newspaper reports, GenBank entries, patents and patent applications cited throughout this application are expressly incorporated herein by reference.

### BRIEF DESCRIPTION OF THE DRAWINGS

FIG. 1A provides individual patient tumor burden percent changes over time. The top of the gray box indicates no change compared to baseline. The bottom of the gray box indicates 50% decrease from baseline. All 23 patients had baseline and at least one post-baseline assessment is presented. Two patients achieved partial response (PR) prior to complete response (CR). (BV, brentuximab vedotin).

FIG. 1B provides individual patient maximum percent reduction in tumor burden from baseline. Complete response (CR) definition is regression to normal size (≦1.5 cm in greatest diameter if >1.5 cm before therapy, or in previously involved nodes 1.1 to 1.5 cm in greatest diameter decreased to ≦1 cm or by more than 75%) and positron-emission tomography (PET) negative. Two patients with CRs met this criteria without 100% decrease. One patients with a partial response achieved 99% decrease but was PET positive. (ASCT, autologous stem-cell transplantation; BV, brentuximab vedotin; HL, Hodgkin lymphoma).

FIG. 2A provides analyses of the PD-L1 and PD-L2 loci and PD-L1 and PD-L2 protein expression in HRS cells. (A) Location and color labeling of bacterial artificial chromosome clones used for the 9p24.1/PD-L1/PD-L2 fluorescence in situ hybridization assay. (B) Representative images of cases with PD-L1/2 copy gain (left panel, 6 green-red [yellow] signals compared to 3 centromeric signals [aqua]) or PD-1/2 amplification (right panel, >3× green-red [yellow] signals compared to centromeric signals [aqua]). (C) PD-L1 (upper panel, brown) and PD-L2 (lower panel, brown) protein expression in Hodgkin Reed-Sternberg (HRS) cells from the same cases as in (B). Arrows indicate malignant cells. PD-L1 is evaluated in conjunction with PAXS to identify PAXS+ HRS cells (upper panel, red). PD-L2 is assessed in association with pSTAT3, which reflects JAK-STAT activation (lower panel, red). Scale bar equals 50 μm.

FIG. 2B provides genetic and immunohistochemical analyses of the PD-L1 and PD-L2 loci, PD-L1 and PD-L2 protein expression, and EBER status. All analyzed cases had structural bases for increased 9p24.1/PD-L1/PD-L2 copy numbers including extra copies of 9p (polysomy 9p), copy gain of PD-L1/PD-L2, or of PD-L1/PD-L2 amplification. Epstein-Barr virus status was evaluated by in situ hybridization of Epstein-Barr virus-encoded messenger RNA (EBERs). (HRS, Hodgkin Reed-Sternberg; IHC, immunohistochemical.)

FIG. 3 provides a schematic of the study design.

FIG. 4 provides analyses of the PD-L1 and PD-L2 Loci and PD-L1 and PD-L2 protein expression in HRS Cells.

FIG. 5 provides analyses of the PD-L1 and PD-L2 Loci and PD-L1 and PD-L2 protein expression in HRS Cells. (A) Location and color labeling of bacterial artificial chromosome clones used for the 9p24.1/PD-L1/PD-L2 fluorescence in situ hybridization assay. (B) Representative images of cases with extra copies of 9p (polysomy 9p, 9 copies of PD-L1, PD-L2 and CEP9 [centromeric probe], first panel), PD-L1/2 copy gain (second panel, 6 green-red [yellow] signals compared to 3 centromeric signals [aqua]), or PD-L1/2 amplification (third and fourth panels, >3× green-red [yellow] signals compared to centromeric signals [aqua]). (C) PD-L1 (upper panel, brown) and PD-L2 (lower panel, brown) protein expression in Hodgkin Reed-Sternberg (HRS) cells from the same cases as in (B). PD-L1 is evaluated in conjunction with PAXS to identify PAXS+ HRS cells (upper panel, red) and PD-L2 is assessed in association with pSTAT3, which reflects JAK-STAT activation (lower panel, red). Scale bar equals 50 μm.

FIG. 6 provides immunohistochemical analyses of PD-L1 and PD-L2 expression in HRS Cells.

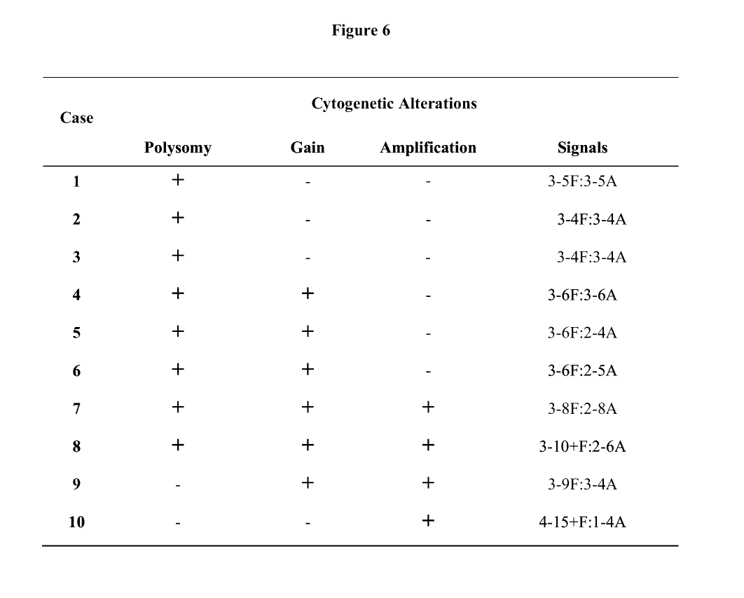

### DETAILED DESCRIPTION OF THE INVENTION

The present invention relates to the unexpectedly superior effects of an anti-PD-1 antibody on treating Hodgkin's lymphoma. In some embodiments, the Hodgkins lymphoma is relapsed Hodgkin's lymphoma. In some embodiments, the Hodgkin's lymphoma is a classical Hodgkin's lymphoma type (cHL) or nodular lymphocyte predominant type. In some embodiments, the nodular lymphocyte predominant type is nodular sclerosing, mixed cellularity, lymphocyte rich or lymphocyte depleted Hodgkin's lymphoma. In some embodiments, the Hodgkins lymphoma is refractory Hodgkin's lymphoma.

In this study, all of the trial patients with cHL and evaluable tumor specimens exhibited concurrent gain of the PD-L1 and PD-L2 loci, increased expression of the PD-1 ligands, and evidence of active JAK-STAT signaling. In this group of patients with relapsed/refractory cHL, the incidence and magnitude of PD-L1/PD-L2 copy gain was higher than in previously reported series of patients with newly diagnosed cHL,^{1, 27} suggesting that this disease-specific genetic alteration may have adverse prognostic significance. Thus, the present invention also relates to the use of biomarkers, for examples PD-L1, PD-L1, a JAK/STAT signaling molecule or Pax5, in determining a suitable Hodgkin's lymphoma patient population for treatment with an anti-PD-1 antibody

### TERMS

In order that the present disclosure can be more readily understood, certain terms are first defined. As used in this application, except as otherwise expressly provided herein, each of the following terms shall have the meaning set forth below. Additional definitions are set forth throughout the application.

“Administering” refers to the physical introduction of a composition comprising a therapeutic agent to a subject, using any of the various methods and delivery systems known to those skilled in the art. Routes of administration for the anti-PD-1 Ab include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Non-parenteral routes include an oral, topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. For example, an adverse event can be associated with activation of the immune system or expansion of immune system cells (e.g., T cells) in response to a treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the Abs can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

An “isolated antibody” refers to an Ab that is substantially free of other Abs having different antigenic specificities (e.g., an isolated Ab that binds specifically to PD-1 is substantially free of Abs that bind specifically to antigens other than PD-1). An isolated Ab that binds specifically to PD-1 can, however, have cross-reactivity to other antigens, such as PD-1 molecules from different species. Moreover, an isolated Ab can be substantially free of other cellular material and/or chemicals.

The term “monoclonal antibody” (“mAb”) refers to a non-naturally occurring preparation of Ab molecules of single molecular composition, i.e., Ab molecules whose primary sequences are essentially identical, and which exhibits a single binding specificity and affinity for a particular epitope. A mAb is an example of an isolated Ab. MAbs can be produced by hybridoma, recombinant, transgenic or other techniques known to those skilled in the art.

A “human” antibody (HuMAb) refers to an Ab having variable regions in which both the framework and CDR regions are derived from human germline immunoglobulin sequences. Furthermore, if the Ab contains a constant region, the constant region also is derived from human germline immunoglobulin sequences. The human Abs of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include Abs in which CDR sequences derived from the germline of another mammalian species, such as a mouse, have been grafted onto human framework sequences. The terms “human” Abs and “fully human” Abs and are used synonymously.

A “humanized antibody” refers to an Ab in which some, most or all of the amino acids outside the CDR domains of a non-human Ab are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the Ab to bind to a particular antigen. A “humanized” Ab retains an antigenic specificity similar to that of the original Ab.

A “chimeric antibody” refers to an Ab in which the variable regions are derived from one species and the constant regions are derived from another species, such as an Ab in which the variable regions are derived from a mouse Ab and the constant regions are derived from a human Ab.

An “anti-antigen” Ab refers to an Ab that binds specifically to the antigen. For example, an anti-PD-1 Ab binds specifically to PD-1 and an anti-CTLA-4 Ab binds specifically to CTLA-4.

An “antigen-binding portion” of an Ab (also called an “antigen-binding fragment”) refers to one or more fragments of an Ab that retain the ability to bind specifically to the antigen bound by the whole Ab.

A “cancer” refers a broad group of various diseases characterized by the uncontrolled growth of abnormal cells in the body. Unregulated cell division and growth divide and grow results in the formation of malignant tumors that invade neighboring tissues and can also metastasize to distant parts of the body through the lymphatic system or bloodstream. In some embodiments, the cancer is Hodgkin's lymphoma. In certain embodiments, the cancer is a classical Hodgkin's lymphoma type or nodular lymphocyte predominant type. In some embodiments, the nodular lymphocyte predominant type is nodular sclerosing, mixed cellularity, lymphocyte rich or lymphocyte depleted Hodgkin's lymphoma. In some embodiments, the Hodgkin's lymphoma is relapsed or refractory.

“Cytotoxic T-Lymphocyte Antigen-4 (CTLA-4) refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“PD-L1 positive” or “PD-L2 positive” as used herein can be interchangeably used with “PD-L1 and/or PD-L2 expression of at least about 1%.” In one embodiment, the PD-L1 and/or PD-L2 expression can be used by any methods known in the art. In another embodiment, the PD-L1 and/or PD-L2 expression is measured by an automated IHC. In another embodiment, the PD-L1 and/or PD-L2 expression is measured by an automated in situ hybridization. A PD-L1 and/or PD-L2 positive tumor can thus have at least about 1%, at least about 2%, at least about 5%, at least about 10%, or at least about 20% of tumor cells expressing PD-L1 as measured by an automated IHC.

“Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1 (PD-L1)” is one of two cell surface glycoprotein ligands for PD-1, the other being “Programmed Death Ligand-2 (PD-L2)”, that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7. The term “PD-L2” as used herein includes human PD-L2 (hPD-L2), variants, isoforms, and species homologs of hPD-L2, and analogs having at least one common epitope with hPD-L2. The complete hPD-L2 sequence can be found under GenBank Accession No. NM_014143.

A “subject” includes any human or nonhuman animal. The term “nonhuman animal” includes, but is not limited to, vertebrates such as nonhuman primates, sheep, dogs, and rodents such as mice, rats and guinea pigs. In some embodiments, the subject is a human. The terms, “subject” and “patient” are used interchangeably herein.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject or prevents further tumor growth. In certain embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount of an anti-cancer agent can inhibit cell growth or tumor growth by at least about 10%, at least about 20%, by at least about 40%, by at least about 60%, or by at least about 80% relative to untreated subjects or, in certain embodiments, relative to patients treated with a standard-of-care therapy. In other embodiments of the invention, tumor regression can be observed and continue for a period of at least about 20 days, at least about 40 days, or at least about 60 days. Notwithstanding these ultimate measurements of therapeutic effectiveness, evaluation of immunotherapeutic drugs must also make allowance for “immune-related” response patterns.

An “immune-related” response pattern refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

A therapeutically effective amount of a drug includes a “prophylactically effective amount,” which is any amount of the drug that, when administered alone or in combination with an anti-neoplastic agent to a subject at risk of developing a cancer (e.g., a subject having a pre-malignant condition) or of suffering a recurrence of cancer, inhibits the development or recurrence of the cancer. In certain embodiments, the prophylactically effective amount prevents the development or recurrence of the cancer entirely. “Inhibiting” the development or recurrence of a cancer means either lessening the likelihood of the cancer“s development or recurrence, or preventing the development or recurrence of the cancer entirely.

The use of the alternative (e.g.,“or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The term “about once a week” as used herein means approximate number, and “about once a week” can include every seven days±two days, i.e., every five days to every nine days. The dosing frequency of “once a week” thus can be every five days, every six days, every seven days, every eight days, or every nine days.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the invention are described in further detail in the following subsections.

### Methods of the Invention

This disclosure provides a method of treating a subject afflicted with Hodgkin's lymphoma, wherein the method comprises administering to the subject a therapeutically effective amount of an Ab or an antigen-binding portion thereof that specifically binds to a PD-1 receptor and inhibits PD-1 activity (“anti-PD-1 antibody”). In another embodiment, the invention includes a method of identifying a Hodgkin's lymphoma subject who responds well to an anti-PD-1 antibody therapy comprising measuring the PD-L1 and/or PD-L2 expression on the tumor and administering to the subject an anti-PD-1 antibody or antigen-binding portion thereof or an anti-PD-L1 antibody or antigen-binding portion thereof. In another embodiment, Pax5 and/or STAT 3 expression on the tumor is measured. In another embodiment, 9p24.1 copy number on the tumor is measured.

The invention also includes methods of determining or identifying a subject afflicted with Hodgkin's lymphoma who is suitable for an anti-PD1 antibody antibody therapy comprising measuring a PD-L1 and/or PD-L2 expression level on the tumor, wherein the tumor has a PD-L1 or PD-L2 expression level of at least about 1% and wherein the subject is administered an anti-PD-1 antibody or antigen-binding portion thereof. In other embodiments, the methods for determining or identifying a subject afflicted with a Hodgkin's lymphoma who is suitable for an anti-PD1 antibody therapy comprises: (i) measuring a PD-L1 and/or PD-L2 expression level on the tumor, wherein the tumor has a PD-L1 and/or PD-L2 expression level of at least about 1% and (ii) administering to the subject a therapeutically effective amount of an anti-PD-1 antibody or antigen-binding portion thereof. In another embodiment, Pax5 and/or STAT 3 expression on the tumor is measured. In another embodiment, 9p24.1 copy number on the tumor is measured.

In certain embodiments, the invention is directed to a method for treating a subject afflicted with a tumor derived from a Hodgkin's lymphoma comprising administering to the subject a therapeutically effective amount of: an anti-PD-1 antibody, wherein the PD-L1 and/or PD-L2 expression level on the tumor is at least about 1%. In other embodiments, the invention includes a method of treating a subject afflicted with a tumor derived from a Hodgkin's lymphoma comprising: (i) measuring a PD-L1 and/or PD-L2 expression level on the tumor, wherein the tumor has a PD-L1 and/or PD-L2 expression level of at least 1% and (ii) administering to the subject a therapeutically effective amount of an anti-PD-1 antibody or antigen-binding portion thereof.

The PD-L1 and/or PD-L2 status of a tumor in a subject can be measured prior to administering any composition or utilizing any method disclosed herein. In one embodiment, the PD-L1 and/or PD-L2 expression level of a tumor is at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20% or greater than at least about 20%. In another embodiment, the PD-L1 and/or PD-L2 status of a tumor is at least about 1%. In other embodiments, the PD-L1 and/or PD-L2 status of the subject is at least about 5%. In a certain embodiment, the PD-L1 and/or PD-L2 status of a tumor is at least about 10%.

In certain embodiments, the therapy of the present invention (e.g., administration of an anti-PD-1 antibody and, optionally, another anti-cancer agent) effectively increases the duration of survival of the subject. In some embodiments, the anti-PD-1 Ab therapy therapy of the present invention increases the duration of survival of the subject in comparison to standard-of-care therapies. In some embodiments, the standard-of-care therapy that the therapy of the present invention is compared to is brentuximab vedotin (BV) or autologous stem cell transplantation. After the administration of an anti-PD-1 antibody therapy, the subject having a PD-L1 and/or PD-L2 positive Hodgkin's lymphoma tumor can exhibit an overall survival of at least about 10 months, at least about 11 months, at least about 12 months, at least about 13 months, at least about 14 months at least about 15 months, at least about 16 months, at least about 17 months, at least about 18 months, at least about 19 months, at least about 20 months, at least about 21 months, at least about 22 months, at least about 23 months, at least about 2 years, at least about 3 years, at least about 4 years, or at least about 5 years after the administration. In a particular embodiment, a subject has a PD-L1 and/or PD-L2 expression level that is at least about 1% and exhibits the overall survival of at least about 17 months. In some embodiments, a subject has a PD-L1 and/or PD-L2 expression level that is at least about 5% and exhibits the overall survival of at least about 18 months. In other embodiments, a subject has a PD-L1 and/or PD-L2 expression level that is at least about 10% and exhibits the overall survival of at least about 19 months.

In other embodiments, the duration of survival or the overall survival of the subject is increased by at least about 1 month, at least about 2 months, at least about 3 months, at least about 4 months, at least about 6 months, or at least about 1 year when compared to another subject treated with only a standard-of-care therapy (e.g., BV). In some embodiments, the overall survival is increased by at least about 4 months, at least about 6 months, at least about 7 months, at least about 8 months, at least about 9 months, at least about 10 months, at least about 11 months, at least about 1 year, at least about 18 months or at least about 2 years when the tumor is PD-L1 and/or PD-L2 positive when compared to another subject treated with only a standard-of-care therapy (e.g., BV). For example, the duration of survival or the overall survival of the subject is increased by at least about 5%, at least about 10%, at least about 15%, at least about 20%, at least about 25%, at least about 30%, at least about 40%, at least about 50% or at least about 75% when compared to another subject treated with only a standard-of-care therapy (e.g., BV).

In certain embodiments, the therapy of the present invention effectively increases the duration of progression free survival of the subject. For example, the progression free survival of the subject is increased by at least about 2 weeks, at least about 1 month, at least about 2 months, at least about 3 months, at least about 4 months, at least about 6 months, or at least about 1 year when compared to another subject treated with only standard-of-care therapy (e.g., BV). For example, the progression free survival of the subject is increased by at least about 2 weeks, at least about 1 month, at least about 2 months, at least about 3 months, at least about 4 months, at least about 6 months, or at least about 1 year when the subject is PD-L1 positive when compared to another subject treated with only standard-of-care therapy (e.g., BV).

In certain embodiments, after the administration of an anti-PD-1 Ab therapy, the subject exhibits an overall response rate of at least about 10%, 20%, 30%, 40%, 45%, or 50% compared to the response rate after administration of a standard-of-care therapy (e.g., BV). In one embodiment, the tumor has a PD-L1 and/or PD-L2 expression level that is at least about 1%, at least about 5% or at least about 10%. In some embodiments, the therapy of the present invention effectively increases the response rate in a group of subjects. For example, the response rate in a group of subjects is increased by at least about 50%, at least about 25%, at least about 10%, at least about 9%, at least about 8%, at least about 7%, at least about 6%, at least about 5%, at least about 4%, at least about 3%, at least about 2%, or at least about 1% when compared to another group of subjects treated with only standard-of-care therapy (e.g., BV). In certain embodiments, the response rate is increased by at least about 2% when compared to another group of subjects treated with only one therapy. In certain embodiments, the median duration of response is increased by at least about 1 month, at least about 2 months, at least about 3 months, at least about 4 months, at least about 6 months or at least about 1 year when the subject is PD-L1 and/or PD-L2 positive when compared to another subject treated with only a standard-of-care therapy (e.g., BV).

In other embodiments, the subject is a human patient. In certain embodiments, the subject is a chemotherapy-naïve patient (e.g., a patient who has not previously received any chemotherapy). In other embodiments, the subject has received another cancer therapy (e.g., a chemotherapy), but is resistant or refractory to such another cancer therapy. In certain embodiments, the PDL1+ and/or PD-L2+ expression level is at least about 1%, at least about 2%, at least about 3%, at least about 4%, at least about 5%, at least about 6%, at least about 7%, at least about 8%, at least about 9%, at least about 10%, at least about 11%, at least about 11%, at least about 12%, at least about 13%, at least about 14%, at least about 15% or at least about 20%. In some embodiments, the subject has Hodgkin's lymphoma and has a PD-L1 and/or PD-L2 expression level of at least about 1%, at least about 5%, or at least about 10%. In one embodiment, the subject has had a prior systematic treatment. In certain embodiments, the subject has had two or more prior systematic treatments.

In order to assess the PD-L1 and/or PD-L2 expression, in one embodiment, a test tissue sample can be obtained from the patient who is in need of the therapy. In another embodiment, the assessment of PD-L1 expression can be achieved without obtaining a test tissue sample. In some embodiments, selecting a suitable patient includes (i) optionally providing a test tissue sample obtained from a patient with cancer of the tissue, the test tissue sample comprising tumor cells and/or tumor-infiltrating inflammatory cells; and (ii) assessing the proportion of cells in the test tissue sample that express PD-L1 and/or PD-L2 on the surface of the cells based on an assessment that the proportion of cells in the test tissue sample that express PD-L1 and/or PD-L2 on the cell surface is higher than a predetermined threshold level.

In any of the methods comprising the measurement of PD-L1 and/or PD-L2 expression in a test tissue sample, however, it should be understood that the step comprising the provision of a test tissue sample obtained from a patient is an optional step. It should also be understood that in certain embodiments the “measuring” or “assessing” step to identify, or determine the number or proportion of, cells in the test tissue sample that express PD-L1 and/or PD-L2 on the cell surface is performed by a transformative method of assaying for PD-L1 expression, for example by performing a reverse transcriptase-polymerase chain reaction (RT-PCR) assay, an IHC assay or a fluorescence in situ hybridization assay. In certain other embodiments, no transformative step is involved and PD-L1 and/or PD-L2 expression is assessed by, for example, reviewing a report of test results from a laboratory. In certain embodiments, the steps of the methods up to, and including, assessing PD-L1 and/or PD-L2 expression provides an intermediate result that may be provided to a physician or other healthcare provider for use in selecting a suitable candidate for the anti-PD-1 antibody therapy. In certain embodiments, the steps that provide the intermediate result is performed by a medical practitioner or someone acting under the direction of a medical practitioner. In other embodiments, these steps are performed by an independent laboratory or by an independent person such as a laboratory technician.

In certain embodiments of any of the present methods, the proportion of cells that express PD-L1 and/or PD-L2 is assessed by performing an assay to determine the presence of PD-L1 and/or PD-L2 RNA. In further embodiments, the presence of PD-L1 and/or PD-L2 RNA is determined by RT-PCR, in situ hybridization or RNase protection. In other embodiments, the proportion of cells that express PD-L1 and/or PD-L2 is assessed by performing an assay to determine the presence of PD-L1 and/or PD-L2 polypeptide. In further embodiments, the presence of PD-L1 polypeptide is determined by immunohistochemistry (IHC), enzyme-linked immunosorbent assay (ELISA), in vivo imaging, or flow cytometry. In some embodiments, PD-L1 and/or PD-L2 expression is assayed by IHC. In some embodiments, PD-L1 and/or PD-L2 expression is assayed by fluorescence in situ hybridization. In other embodiments of all of these methods, cell surface expression of PD-L1 In some embodiments, PD-L1 and/or PD-L2 expression is assayed by IHC. is assayed using, e.g., IHC or in vivo imaging. In certain embodiments, a 9p24.1 copy number is determined by fluorescent in situ hybridization. In another embodiment, Pax5 and/or STAT3 expression are determined by IHC.

Imaging techniques have provided important tools in cancer research and treatment. Recent developments in molecular imaging systems, including positron emission tomography (PET), single-photon emission computed tomography (SPECT), fluorescence reflectance imaging (FRI), fluorescence-mediated tomography (FMT), bioluminescence imaging (BLI), laser-scanning confocal microscopy (LSCM) and multiphoton microscopy (MPM), will likely herald even greater use of these techniques in cancer research. Some of these molecular imaging systems allow clinicians to not only see where a tumor is located in the body, but also to visualize the expression and activity of specific molecules, cells, and biological processes that influence tumor behavior and/or responsiveness to therapeutic drugs (Condeelis and Weissleder, 2010). Ab specificity, coupled with the sensitivity and resolution of PET, makes immunoPET imaging particularly attractive for monitoring and assaying expression of antigens in tissue samples (McCabe and Wu, 2010; Olafsen et al., 2010). In certain embodiments of any of the present methods, PD-L1 and/or PD-L2 expression is assayed by immunoPET imaging. In certain embodiments of any of the present methods, the proportion of cells in a test tissue sample that express PD-L1 and/or PD-L2 is assessed by performing an assay to determine the presence of PD-L1 and/or PD-L2 polypeptide on the surface of cells in the test tissue sample. In certain embodiments, the test tissue sample is a FFPE tissue sample. In other embodiments, the presence of PD-L1 and/or PD-L2 polypeptide is determined by IHC assay. In further embodiments, the IHC assay is performed using an automated process. In some embodiments, the IHC assay is performed using an anti-PD-L1 mAb to bind to the PD-L1 polypeptide or using an anti-PD-L2 mAb to bind to the PD-L2 polypeptide.

In one embodiment of the present methods, an automated IHC method is used to assay the expression of PD-L1 and/or PD-L2 on the surface of cells in FFPE tissue specimens. This disclosure provides methods for detecting the presence of human PD-L1 and/or PD-L2 antigen in a test tissue sample, or quantifying the level of human PD-L1 and/or PD-L2 antigen or the proportion of cells in the sample that express the antigen, which methods comprise contacting the test sample, and a negative control sample, with a mAb that specifically binds to human PD-L1, under conditions that allow for formation of a complex between the Ab or portion thereof and human PD-L1 and/or PD-L2. In certain embodiments, the test and control tissue samples are FFPE samples. The formation of a complex is then detected, wherein a difference in complex formation between the test sample and the negative control sample is indicative of the presence of human PD-L1 antigen in the sample. Various methods are used to quantify PD-L1 and/or PD-L2 expression.

In a particular embodiment, the automated IHC method comprises: (a) deparaffinizing and rehydrating mounted tissue sections in an autostainer; (b) retrieving antigen using a decloaking chamber and pH 6 buffer, heated to 110° C. for 10 min; (c) setting up reagents on an autostainer; and (d) running the autostainer to include steps of neutralizing endogenous peroxidase in the tissue specimen; blocking non-specific protein-binding sites on the slides; incubating the slides with primary Ab; incubating with a postprimary blocking agent; incubating with NovoLink Polymer; adding a chromogen substrate and developing; and counterstaining with hematoxylin.

For assessing PD-L1 and/or PD-L2 expression in tumor tissue samples, a pathologist examines the number of membrane PD-L1+ and/or PD-L2 tumor cells in each field under a microscope and mentally estimates the percentage of cells that are positive, then averages them to come to the final percentage. The different staining intensities are defined as 0/negative, 1+/weak, 2+/moderate, and 3+/strong. Typically, percentage values are first assigned to the 0 and 3+ buckets, and then the intermediate 1+ and 2+ intensities are considered. For highly heterogeneous tissues, the specimen is divided into zones, and each zone is scored separately and then combined into a single set of percentage values. The percentages of negative and positive cells for the different staining intensities are determined from each area and a median value is given to each zone. A final percentage value is given to the tissue for each staining intensity category: negative, 1+, 2+, and 3+. The sum of all staining intensities needs to be 100%.

Staining is also assessed in tumor-infiltrating inflammatory cells such as macrophages and lymphocytes. In most cases macrophages serve as an internal positive control since staining is observed in a large proportion of macrophages. While not required to stain with 3+ intensity, an absence of staining of macrophages should be taken into account to rule out any technical failure. Macrophages and lymphocytes are assessed for plasma membrane staining and only recorded for all samples as being positive or negative for each cell category. Staining is also characterized according to an outside/inside tumor immune cell designation. “Inside” means the immune cell is within the tumor tissue and/or on the boundaries of the tumor region without being physically intercalated among the tumor cells. “Outside” means that there is no physical association with the tumor, the immune cells being found in the periphery associated with connective or any associated adjacent tissue.

In certain embodiments of these scoring methods, the samples are scored by two pathologists operating independently, and the scores are subsequently consolidated. In certain other embodiments, the identification of positive and negative cells is scored using appropriate software.

A histoscore is used as a more quantitative measure of the IHC data. The histoscore is calculated as follows:

Histoscore=[(% tumor×1 (low intensity))+(% tumor×2 (medium intensity))+(% tumor×3 (high intensity)]

To determine the histoscore, the pathologist estimates the percentage of stained cells in each intensity category within a specimen. Because expression of most biomarkers is heterogeneous the histoscore is a truer representation of the overall expression. The final histoscore range is 0 (no expression) to 300 (maximum expression).

An alternative means of quantifying PD-L1 and/or PD-L2 expression in a test tissue sample IHC is to determine the adjusted inflammation score (AIS) score defined as the density of inflammation multiplied by the percent PD-L1 and/or PD-L2 expression by tumor-infiltrating inflammatory cells (Taube et al., 2012).

The present methods can treat any type of Hodgkins lymphoma. In certain embodiments, the Hodgkins lymphoma is cHL or nodular lymphocyte predominant type Hodgkin's lymphoma. In certain embodiments, the nodular lymphocyte predominant type Hodgkin's lymphoma is a Hodgkins lymphoma selected from the group consisting of nodular sclerosing, mixed cellularity, lymphocyte rich and lymphocyte depleted.

### Anti-PD-1 Antibodies and Anti-PD-L1 Antibodies

Anti-PD-1 Abs suitable for use in the disclosed methods are Abs that bind to PD-1 with high specificity and affinity, block the binding of PD-L1, and inhibit the immunosuppressive effect of the PD-1 signaling pathway. In any of the therapeutic methods disclosed herein, an anti-PD-1 or anti-PD-L1 “antibody” includes an antigen-binding portion that binds to the PD-1 or PD-L1 receptor, respectively, and exhibits the functional properties similar to those of whole Abs in inhibiting ligand binding and upregulating the immune system. In certain embodiments, the anti-PD-1 Ab or antigen-binding portion thereof cross-competes with nivolumab for binding to human PD-1. In other embodiments, the anti-PD-L1 antibody or antigen-binding fragment thereof competes for binding with BMS-936559, MPDL3280A, MEDI4736 or MSB0010718C for binding to human PD-L1.

In other embodiments, the anti-PD-1 Ab, or anti-PD-L1 Ab, or antigen-binding portions thereof is a chimeric, humanized or human monoclonal Ab or a portion thereof. In certain embodiments for treating a human subject, the Ab is a humanized Ab. In other embodiments for treating a human subject, the Ab is a human Ab. Abs of an IgG1, IgG2, IgG3 or IgG4 isotype can be used.

In certain embodiments, the anti-PD-1 Ab, or anti-PD-L1 Ab, or antigen-binding portions thereof comprises a heavy chain constant region which is of a human IgG1 or IgG4 isotype. In certain other embodiments, the sequence of the IgG4 heavy chain constant region of the anti-PD-1 Ab, or anti-PD-L1 Ab, or antigen-binding portions thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al., 2014). In yet other embodiments, the Ab comprises a light chain constant region which is a human kappa or lambda constant region. In other embodiments, the anti-PD-1 Ab, or anti-PD-L1 Ab, or antigen-binding portions thereof is a mAb or an antigen-binding portion thereof.

HuMAbs that bind specifically to PD-1 with high affinity have been disclosed in U.S. Pat. No. 8,008,449. Other anti-PD-1 mAbs have been described in, for example, U.S. Pat. Nos. 6,808,710, 7,488,802, 8,168,757 and 8,354,509, and PCT Publication No. WO 2012/145493. Each of the anti-PD-1 HuMAbs disclosed in U.S. Pat. No. 8,008,449 has been demonstrated to exhibit one or more of the following characteristics: (a) binds to human PD-1 with a K_{D} of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates Ab responses; and (j) inhibits tumor cell growth in vivo. Anti-PD-1 Abs usable in the present invention include mAbs that bind specifically to human PD-1 and exhibit at least one, in some embodiments, at least five, of the preceding characteristics. In some embodiments, the anti-PD-1 Ab is nivolumab. In one embodiment, the anti-PD-1 Ab is pembrolizumab.

In one embodiment, the anti-PD-1 Ab is nivolumab. Nivolumab (also known as “OPDIVO®”; formerly designated 5C4, BMS-936558, MDX-1106, or ONO-4538) is a fully human IgG4 (S228P) PD-1 immune checkpoint inhibitor Ab that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In another embodiment, the anti-PD-1 Ab is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587. Pembrolizumab has been approved by the FDA for the treatment of relapsed or refractory melanoma.

In other embodiments, the anti-PD-1 Ab is MEDI0608 (formerly AMP-514), which is a monoclonal antibody. MEDI0608 is described, for example, in U.S. Pat. No. 8,609,089B2.

In some embodiments, the anti-PD-1 antibody is Pidilizumab (CT-011), which is a humanized monoclonal antibody. Pidilizumab is described in U.S. Pat. No. 8,686,119 B2 or WO 2013/014668 A1.

Anti-PD-1 Abs usable in the disclosed methods also include isolated Abs that bind specifically to human PD-1 and cross-compete for binding to human PD-1 with nivolumab (see, e.g., U.S. Pat. Nos. 8,008,449 and 8,779,105; WO 2013/173223). The ability of Abs to cross-compete for binding to an antigen indicates that these Abs bind to the same epitope region of the antigen and sterically hinder the binding of other cross-competing Abs to that particular epitope region. These cross-competing Abs are expected to have functional properties very similar those of nivolumab by virtue of their binding to the same epitope region of PD-1. Cross-competing Abs can be readily identified based on their ability to cross-compete with nivolumab in standard PD-1 binding assays such as Biacore analysis, ELISA assays or flow cytometry (see, e.g., WO 2013/173223).

In certain embodiments, the Abs that cross-compete for binding to human PD-1 with, or bind to the same epitope region of human PD-1 as, nivolumab are mAbs. For administration to human subjects, these cross-competing Abs are chimeric Abs, or humanized or human Abs. Such chimeric, humanized or human mAbs can be prepared and isolated by methods well known in the art.

Anti-PD-1 Abs usable in the methods of the disclosed invention also include antigen-binding portions of the above Abs. It has been amply demonstrated that the antigen-binding function of an Ab can be performed by fragments of a full-length Ab. Examples of binding fragments encompassed within the term “antigen-binding portion” of an Ab include (i) a Fab fragment, a monovalent fragment consisting of the V_{L}, V_{H}, C_{L} and C_{H1} domains; (ii) a F(ab″)₂ fragment, a bivalent fragment comprising two Fab fragments linked by a disulfide bridge at the hinge region; (iii) a Fd fragment consisting of the V_{H} and C_{H1} domains; and (iv) a Fv fragment consisting of the V_{L} and V_{H} domains of a single arm of an Ab.

In certain embodiments of any of the therapeutic methods described herein comprising administration of an anti-PD-1 Ab, the anti-PD-1 Ab is nivolumab. In other embodiments, the anti-PD-1 Ab is pembrolizumab. In other embodiments, the anti-PD-1 Ab is chosen from the human antibodies 17D8, 2D3, 4H1, 4A11, 7D3 and 5F4 described in U.S. Pat. No. 8,008,449. In still other embodiments, the anti-PD-1 Ab is MEDI0608 (formerly AMP-514), AMP-224, or Pidilizumab (CT-011).

In certain embodiments, an anti-PD-1 antibody used in the methods may be replaced with another PD-1 or anti-PD-L1 antagonist. For example, because an anti-PD-L1 antibody prevents interaction between PD-1 and PD-L1, thereby exerting similar effects to the signaling pathway of PD-1, an anti-PD-L1 antibody may replace the use of an anti-PD-1 antibody in the methods disclosed herein. Therefore, in one embodiment, the present invention is directed to a method for treating a subject afflicted with a Hodgkin's lymphoma comprising administering to the subject a therapeutically effective amount an anti-PD-L1 antibody. In certain embodiments, the anti-PD-L1 Ab is BMS-936559 (formerly 12A4 or MDX-1105) (see, e.g., U.S. Pat. No. 7,943,743; WO 2013/173223). In other embodiments, the anti-PD-L1 Ab is MPDL3280A (also known as RG7446) (see, e.g., Herbst; U.S. Pat. No. 8,217,149), MEDI4736 (also called Durvalumab; Khleif, 2013, See U.S. Pat. No. 8,779,108 or US 2014/0356353, filed May 6, 2014), or MSB0010718C (also called Avelumab; See US 2014/0341917).

Combination Therapies with Anti-PD-1 or Anti-PD-L1 Antibodies

In certain embodiments, an anti-PD-1 antibody is administered in combination with one or more other anti-cancer agents. In some embodiments, the other anti-cancer agent is any anti-cancer agent described herein or known in the art. In certain embodiments, the other anti-cancer agent is an anti-CTLA-4 antibody. In one embodiment, the other anti-cancer agent is BV. In certain embodiments, the other anti-cancer agent is an EGFR-targeted tyrosine kinase inhibitor (TKI). In one embodiment, the other anti-cancer agent is an anti-VEGF antibody. In other embodiments, the anti-cancer agent is a platinum agent (e.g., cisplatin, carboplatin), a mitotic inhibitor (e.g., paclitaxel, albumin-bound paclitaxel, docetaxel, taxotere, docecad), a fluorinated Vinca alkaloid (e.g., vinflunine, javlor), vinorelbine, vinblastine, etoposide, or pemetrexed gemcitabin. In one embodiment, the other anti-cancer agent is 5-flurouracil (5-FU). In certain embodiments, the other anti-cancer agent is any other anti-cancer agent known in the art. In some embodiments, two or more additional anti-cancer agents are administered in combination with the anti-PD-1. In some embodiments, the PD-1 antibody is combined with an autologous stem cell transplant or radiation.

### Anti-CTLA-4 Antibodies

Anti-CTLA-4 antibodies of the instant invention bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

HuMAbs that bind specifically to CTLA-4 with high affinity have been disclosed in U.S. Pat. Nos. 6,984,720 and 7,605,238. Other anti-PD-1 mAbs have been described in, for example, U.S. Pat. Nos. 5,977,318, 6,051,227, 6,682,736, and 7,034,121. The anti-PD-1 HuMAbs disclosed in U.S. Pat. Nos. 6,984,720 and 7,605,238 have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 1011 M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹, and (d) inhibits the binding of CTLA-4 to B7-1 (CD80) and B7-2 (CD86). Anti-CTLA-4 Abs usable in the present invention include mAbs that bind specifically to human CTLA-4 and exhibit at least one, and, in one embodiment, at least three of the preceding characteristics. An exemplary clinical anti-CTLA-4 Ab is the human mAb 10D1 (now known as ipilimumab and marketed as YERVOY®) as disclosed in U.S. Pat. No. 6,984,720. Ipilimumab is an anti-CTLA-4 Ab for use in the methods disclosed herein. Another anti-CTLA-4 Ab usable in the present methods is tremelimumab.

An exemplary clinical anti-CTLA-4 Ab is the human mAb 10D1 (now known as ipilimumab and marketed as YERVOY®) as disclosed in U.S. Pat. No. 6,984,720. Ipilimumab is an anti-CTLA-4 Ab for use in the methods disclosed herein. Ipilimumab is a fully human, IgG1 monoclonal Ab that blocks the binding of CTLA-4 to its B7 ligands, thereby stimulating T cell activation and improving overall survival (OS) in patients with advanced melanoma.

Another anti-CTLA-4 Ab useful for the present methods is tremelimumab (also known as CP-675,206). Tremelimumab is human IgG2 monoclonal anti-CTLA-4 antibody. Tremelimumab is described in WO/2012/122444, U.S. Publ. No. 2012/263677, or WO Publ. No. 2007/113648 A2.

Anti-CTLA-4 Abs usable in the disclosed methods also include isolated Abs that bind specifically to human PD-1 and cross-compete for binding to human CTLA-4 with ipilimumab or tremelimumab or bind to the same epitope region of human CTLA-4 as ipilimumab or tremelimumab. In certain embodiments, the Abs that cross-compete for binding to human CTLA-4 with, or bind to the same epitope region of human PD-1 as does ipilimumab or tremelimumab, are Abs comprising a heavy chain of the human IgG1 isotype. For administration to human subjects, these cross-competing Abs are chimeric Abs, or humanized or human Abs. Usable anti-CTLA-4 Abs also include antigen-binding portions of the above Abs such as Fab, F(ab″)₂, Fd or Fv fragments.

Ipilimumab (YERVOY®) is a fully human, IgG1 monoclonal Ab that blocks the binding of CTLA-4 to its B7 ligands, thereby stimulating T cell activation and improving overall survival (OS) in patients with advanced melanoma (Hodi et al., 2010). Concurrent therapy with nivolumab and ipilimumab in a Phase 1 clinical trial produced rapid and deep tumor regression in a substantial proportion of patients with advanced melanoma, and was significantly more effective than either Ab alone (Wolchok et al., 2013; WO 2013/173223). However, it was hitherto not known whether this combination of immunoregulatory Abs would be similarly effective in other tumor types.

### Chemotherapy and Platinum-Based Chemotherapy

In some embodiments, the anti-PD1 antibody is administered in combination with any chemotherapy known in the art. In certain embodiments, the chemotherapy is a platinum based-chemotherapy. Platinum-based chemotherapies are coordination complexes of platinum. In some embodiments, the platinum-based chemotherapy is a platinum-doublet chemotherapy. In one embodiment, the chemotherapy is administered at the approved dose for the particular indication. In other embodiments, the chemotherapy is administered at any dose disclosed herein. In some embodiments, the platinum-based chemotherapy is cisplatin, carboplatin, oxaliplatin, satraplatin, picoplatin, Nedaplatin, Triplatin, Lipoplatin, or combinations thereof. In certain embodiments, the platinum-based chemotherapy is any other platinum-based chemotherapy known in the art. In some embodiments, the chemotherapy is the nucleotide analog gemcitabine. In an embodiment, the chemotherapy is a folate antimetabolite. In an embodiment, the folate antimetabolite is pemetrexed. In certain embodiments the chemotherapy is a taxane. In other embodiments, the taxane is paclitaxel. In other embodiments, the chemotherapy is a nucleoside analog. In certain embodiments, the chemotherapy is doxorubicin, bleomycin or vinblastine, dacarbazine. In one embodiment, the nucleoside analog is gemcitabine. In certain embodiments, the chemotherapy is ifosfamide, carboplatin or etoposide. In one embodiment, the nucleoside analog is gemcitabine. In some embodiments, the chemotherapy is any other chemotherapy known in the art. In certain embodiments, at least one, at least two or more chemotherapeutic agents are administered in combination with the anti-PD1 antibody. In some embodiments, the anti-PD1 antibody is administered in combination with gemcitabine and cisplatin. In some embodiments, the anti-PD1 antibody is administered in combination with pemetrexed and cisplatin. In certain embodiments, the anti-PD1 antibody is administered in combination with gemcitabine and pemetrexed. In one embodiment, the anti-PD1 antibody is administered in combination with paclitaxel and carboplatin. In an embodiment, an anti-CTLA-4 antibody is additionally administered.

### Tyrosine Kinase Inhibitors

In certain embodiments, the anti-PD-1 antibody is administered in combination with a tyrosine kinase inhibitor. In certain embodiments, the tyrosine kinase inhibitor is gefitinib, erlotinib, combinations thereof or any other tyrosine kinase inhibitor known in the art. In some embodiments, the tyrosine kinase inhibitor act on the epidermal growth factor receptor (EGFR). In an embodiment, an anti-CTLA-4 antibody is additionally administered.

### Immunotherapy of Hodgkin's Lymphoma

A clear need exists for effective agents for patients who have progressed on multiple lines of targeted therapy, as well as for therapies that extend survival for longer periods beyond the current standard treatments. Newer approaches involving immunotherapy, especially blockade of immune checkpoints including the CTLA-4, PD-1, and PD-L1 inhibitory pathways, have recently shown promise. In certain embodiments, immunotherapy involving blockade of immune checkpoints is administered as a monotherapy. In other embodiments, immunotherapy involving blockade of immune checkpoints is administered in combination with other therapies. In addition, dual checkpoint blockade strategies, such as those combining anti-PD-1 and anti-CTLA-4 have proven to be highly effective in treating melanoma (Wolchok et al, 2013; WO 2013/173223), and other combinations including anti-PD-L1, anti-LAG-3, or anti-KIR, are being tested to increase the proportion and durability of tumor responses. By analogy to melanoma, Hodgkin's lymphoma patients can to benefit either from the combination of different immunotherapeutic drugs or the combination of such drugs with targeted agents or other treatments including, surgery, radiation, standard cancer chemotherapies, autologous stem cell transplants or vaccines. However, surprising and unexpected complications have sometimes been observed when immunotherapeutics are combined with other anti-cancer agents. Thus, the combination of immunotherapy (including an immune checkpoint inhibitor drug such as an anti-CTLA-4 or anti-PD-1 Ab) with other anti-cancer agents is unpredictable and must be carefully assessed for safety as well as efficacy in clinical trials.

### Pharmaceutical Compositions and Dosages

Therapeutic agents of the present invention can be constituted in a composition, e.g., a pharmaceutical composition containing an Ab and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. In one embodiment, the carrier for a composition containing an Ab is suitable for intravenous, intramuscular, subcutaneous, parenteral, spinal or epidermal administration (e.g., by injection or infusion), whereas the carrier for a composition containing a TKI is suitable for non-parenteral, e.g., oral, administration. A pharmaceutical composition of the invention can include one or more pharmaceutically acceptable salts, anti-oxidant, aqueous and non-aqueous carriers, and/or adjuvants such as preservatives, wetting agents, emulsifying agents and dispersing agents.

Dosage regimens are adjusted to provide the optimum desired response, e.g., a maximal therapeutic response and/or minimal adverse effects. For administration of an anti-PD-1 Ab, as a monotherapy or in combination with another anti-cancer agent, the dosage can range from about 0.01 to about 20 mg/kg, about 0.1 to about 10 mg/kg, about 0.1 to about 5 mg/kg, about 1 to about 5 mg/kg, about 2 to about 5 mg/kg, about 7.5 to about 12.5 mg/kg, or about 0.1 to about 30 mg/kg of the subject”s body weight. For example, dosages can be about 0.1, about 0.3, about 1, about 2, about 3, about 5 or about 10 mg/kg body weight, or about 0.3, about 1, about 2, about 3, or about 5 mg/kg body weight. The dosing schedule is typically designed to achieve exposures that result in sustained receptor occupancy (RO) based on typical pharmacokinetic properties of an Ab. An exemplary treatment regime entails administration about once per week, about once every 2 weeks, about once every 3 weeks, about once every 4 weeks, about once a month, about once every 3-6 months or longer. In certain embodiments, an anti-PD-1 Ab such as nivolumab is administered to the subject about once every 2 weeks. In other embodiments, the Ab is administered about once every 3 weeks. The dosage and scheduling can change during a course of treatment. For example, a dosing schedule for anti-PD-1 monotherapy can comprise administering the Ab: (i) about every 2 weeks in about 6-week cycles; (ii) about every 4 weeks for about six dosages, then about every three months; (iii) about every 3 weeks; (iv) about 3-about 10 mg/kg once followed by about 1 mg/kg every about 2-3 weeks. Considering that an IgG4 Ab typically has a half-life of 2-3 weeks, a dosage regimen for an anti-PD-1 Ab of the invention comprises about 0.3-1 about 0 mg/kg body weight, 1-5 mg/kg body weight, or about 1-about 3 mg/kg body weight via intravenous administration, with the Ab being given every about 14-21 days in up to about 6-week or about 12-week cycles until complete response or confirmed progressive disease. In certain embodiments, an anti-PD-1 monotherapy is administered at 3 mg/kg every 2 weeks until progressive disease or unacceptable toxicity. In some embodiments, the antibody treatment, or any combination treatment disclosed herein, is continued for at least about 1 month, at least about 3 months, at least about 6 months, at least about 9 months, at least about 1 year, at least about 18 months, at least about 24 months, at least about 3 years, at least about 5 years, or at least about 10 years.

When used in combinations with other cancer agents, the dosage of an anti-PD-1 Ab can be lowered compared to the monotherapy dose. For example, a dosage of nivolumab that is significantly lower than the typical about 3 mg/kg about every 3 weeks, for instance about 0.1 mg/kg or less about every 3 or 4 weeks, is regarded as a subtherapeutic dosage. Receptor-occupancy data from 15 subjects who received 0.3 mg/kg to 10 mg/kg dosing with nivolumab indicate that PD-1 occupancy appears to be dose-independent in this dose range. Across all doses, the mean occupancy rate was 85% (range, 70% to 97%), with a mean plateau occupancy of 72% (range, 59% to 81%) (Brahmer et al., 2010). Thus, 0.3 mg/kg dosing can allow for sufficient exposure to lead to maximal biologic activity.

Although higher nivolumab monotherapy dosing up to about 10 mg/kg every two weeks has been achieved without reaching the maximum tolerated does (MTD), the significant toxicities reported in other trials of checkpoint inhibitors plus anti-angiogenic therapy (see, e.g., Johnson et al., 2013; Rini et al., 2011) support the selection of a nivolumab dose lower than 10 mg/kg.

In certain embodiments, the dose of an anti-PD-1 antibody is a fixed dose in a pharmaceutical composition. In other embodiments, the method of the present invention can be used with a flat dose (a dose given to a patient irrespective of the body weight of the patient). For example, a flat dose of a nivolumab can be about 240 mg. For example, a flat dose of pembrolizumab can be about 200 mg.

Ipilimumab (YERVOY®) is approved for the treatment of melanoma at 3 mg/kg given intravenously once every 3 weeks for 4 doses. Thus, in some embodiments, about 3 mg/kg is the highest dosage of ipilimumab used in combination with the anti-PD-1 Ab though, in certain embodiments, an anti-CTLA-4 Ab such as ipilimumab can be dosed within the range of about 0.3 to about 10 mg/kg, about 0.5 to about 10 mg/kg, about 0.5 to about 5 mg/kg, or about 1 to about 5 mg/kg. body weight about every two or three weeks when combined with nivolumab. In other embodiments, ipilimumab is administered on a different dosage schedule from nivolumab. In some embodiments, ipilimumab is administered about every week, about every two weeks, about every three weeks, about every 4 weeks, about every five weeks, about every six weeks, about every seven weeks, about every eight weeks, about every nine weeks, about every ten weeks, about every eleven weeks, about every twelve weeks or about every fifteen weeks. A dosage of ipilimumab that is significantly lower than the approved about 3 mg/kg about every 3 weeks, for instance about 0.3 mg/kg or less about every 3 or 4 weeks, is regarded as a subtherapeutic dosage. It has been shown that combination dosing of nivolumab at 3 mg/kg and ipilimumab at 3 mg/kg exceeded the MTD in a melanoma population, whereas a combination of nivolumab at 1 mg/kg plus ipilimumab at 3 mg/kg or nivolumab at 3 mg/kg plus ipilimumab at 1 mg/kg was found to be tolerable in melanoma patients (Wolchok et al., 2013). Accordingly, although nivolumab is tolerated up to 10 mg/kg given intravenously every 2 weeks, in certain embodiments doses of the anti-PD-1 Ab do not exceed about 3 mg/kg when combined with ipilimumab. In certain embodiments, based on risk-benefit and PK-PD assessments, the dosage used comprises a combination of nivolumab at about 1 mg/kg plus ipilimumab at about 3 mg/kg, nivolumab at about 3 mg/kg plus ipilimumab at about 1 mg/kg, or nivolumab at about 3 mg/kg plus ipilimumab at about 3 mg/kg is used, each administered at a dosing frequency of once about every 2-4 weeks, in certain embodiments, once about every 2 weeks or once about every 3 weeks. In certain other embodiments, nivolumab is administered at a dosage of about 0.1, about 0.3, about 1, about 2, about 3 or about 5 mg/kg in combination with ipilimumab administered at a dosage of about 0.1, about 0.3, about 1, about 2, about 3 or about 5 mg/kg, once about every 2 weeks, once about every 3 weeks, or once about every 4 weeks.

In certain embodiments, the combination of an anti-PD-1 Ab and an anti-CTLA-4 Ab is administered intravenously to the subject in an induction phase about every 2 or 3 weeks for 1, 2, 3 or 4 administrations. In certain embodiments, the combination of nivolumab and ipilimumab is administered intravenously in the induction phase about every 2 weeks or about every 3 weeks for about 4 administrations. The induction phase is followed by a maintenance phase during which only the anti-PD-1 Ab is administered to the subject at a dosage of about 0.1, about 0.3, about 1, about 2, about 3, about 5 or about 10 mg/kg about every two or three weeks for as long as the treatment proves efficacious or until unmanageable toxicity or disease progression occurs. In certain embodiments, nivolumab is administered during the maintenance phase at a dose of about 3 mg/kg body about every 2 weeks.

In certain embodiments, the anti-PD-1 antibody and the anti-CTLA-4 antibody are formulated as a single composition, wherein the dose of the anti-PD1 antibody and the dose of the anti-CTLA-4 antibody are combined at a ratio of 1:50, 1:40, 1:30, 1:20, 1:10. 1:5, 1:3, 1:1, 3:1, 5:1, 10:1, 20:1, 30:1, 40:1, or 50:1. In other embodiments, the dose of the anti-CTLA-4 antibody is a fixed dose. In certain embodiments, the dose of the anti-CTLA-4 antibody is a flat dose, which is given to a patient irrespective of the body weight. In a specific embodiment, the flat dose of the anti-CTLA-4 antibody is about 80 mg.

For combination of nivolumab with other anti-cancer agents, these agents are administered at their approved dosages. Treatment is continued as long as clinical benefit is observed or until unacceptable toxicity or disease progression occurs. Nevertheless, in certain embodiments, the dosages of these anti-cancer agents administered are significantly lower than the approved dosage, i.e., a subtherapeutic dosage, of the agent is administered in combination with the anti-PD-1 Ab. The anti-PD-1 Ab can be administered at the dosage that has been shown to produce the highest efficacy as monotherapy in clinical trials, e.g., about 3 mg/kg of nivolumab administered once about every three weeks (Topalian et al., 2012a; Topalian et al., 2012), or at a significantly lower dose, i.e., at a subtherapeutic dose. In certain embodiments, the anti-PD-1 Ab is administered at about 3 mg/kg once about every two weeks.

Dosage and frequency vary depending on the half-life of the Ab in the subject. In general, human Abs show the longest half-life, followed by humanized Abs, chimeric Abs, and nonhuman Abs. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, or until the patient shows partial or complete amelioration of symptoms of disease. Thereafter, the patient can be administered a prophylactic regime.

Actual dosage levels of the active ingredient or ingredients in the pharmaceutical compositions of the present invention can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present invention can be administered via one or more routes of administration using one or more of a variety of methods well known in the art. As will be appreciated by the skilled artisan, the route and/or mode of administration will vary depending upon the desired results.

### Kits

Also within the scope of the present invention are kits comprising an anti-PD-1 Ab and, optionally, another anti-cancer agent for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a Hodgkin's lymphoma, the kit comprising: (a) a dosage ranging from about 0.1 to about 10 mg/kg body weight of an anti-cancer agent which is an Ab or an antigen-binding portion thereof that specifically binds to the PD-1 receptor and inhibits PD-1 activity; and, optionally, (b) a dosage of another anti-cancer agent which is any anti-cancer agent described herein, including: (i) a platinum-based doublet chemotherapy; (ii) a tyrosine kinase inhibitor; or (iii) a dosage ranging from about 0.1 to about 10 mg/kg body weight of an antibody or an antigen-binding portion thereof that specifically binds to and inhibits CTLA-4; and (c) instructions for using the anti-PD-1 Ab and, optionally, the other anti-cancer agent in any of the therapy methods disclosed herein. In certain embodiments, the anti-PD-1, the anti-CTLA-4 Ab and/or the TKI can be co-packaged in unit dosage form. In certain embodiments for treating human patients, the kit comprises an anti-human PD-1 Ab disclosed herein, e.g., nivolumab or pembrolizumab. In other embodiments, the kit comprises an anti-human CTLA-4 Ab disclosed herein, e.g., ipilimumab or tremelimumab.

The present invention is further illustrated by the following example, which should not be construed as further limiting. The contents of all references cited throughout this application are expressly incorporated herein by reference.

### Example 1

### Treatment of Hodgkin's Lymphoma with Nivolumab

### Methods

### Patients

To be eligible for participation in this study, patients had to be at least 18 years of age, have histologically confirmed evidence of relapsed or refractory Hodgkin's lymphoma with at least one lesion measuring More than 1.5 cm (as defined by the Revised Response Criteria for Malignant Lymphomas (18) an Eastern Cooperative Oncology Group (ECOG)(19) performance-status score of 0 or 1 (on a scale from 0 to 5, with 0 indicating no symptoms and higher scores indicating increasing disability), previous treatment with at least one chemotherapy regimen, and no autologous stem-cell transplantation within the previous 100 days. Key exclusion criteria were a history of cancer involving the central nervous system, a history of or active autoimmune disease, a concomitant second cancer, and previous organ allograft or allogeneic bone marrow transplantation.

### Patients

This study (ClinicalTrials.gov identifier: NCT01592370) enrolled patients with relapsed/refractory cHL who met the following eligibility criteria: at least 18 years of age; histologically confirmed evidence of relapsed/refractory cHL with at least one measureable lesion >1.5 cm defined by the Revised Response Criteria for Malignant Lymphomas²²; Eastern Cooperative Oncology Group (ECOG) performance status of 0 or 1; and prior treatment with at least one chemotherapy regimen and no autologous stem-cell transplantation (ASCT) within the previous 100 days. Key exclusion criteria included history of central nervous system involvement by malignancy, concomitant second malignancy, and prior organ allograft or allogeneic bone marrow transplantation.

### Study Design

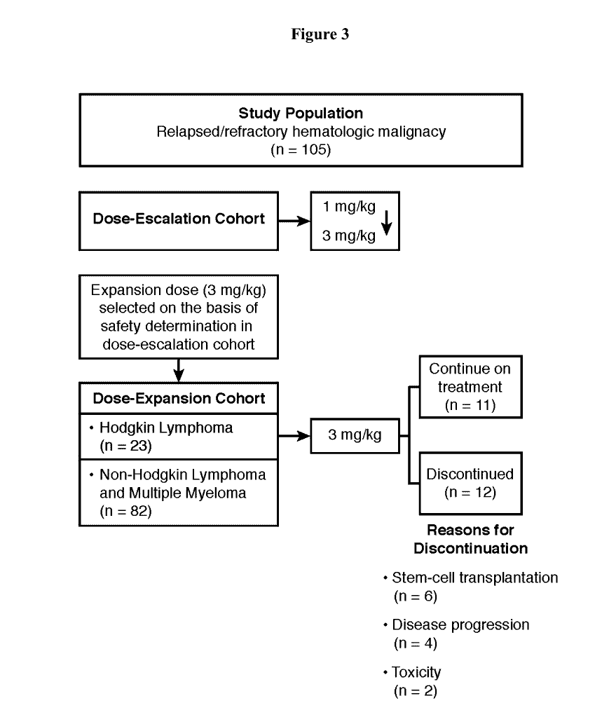

This phase 1 study consisted of dose-escalation and expansion cohorts (see FIG. 3). In the dose-escalation cohort, patients with relapsed/refractory hematologic malignancies were treated with nivolumab 1 mg/kg escalating to 3 mg/kg. As the maximum tolerated dose was not reached, the 3-mg/kg dose was chosen for the expansion cohorts. Patients with relapsed/refractory cHL received nivolumab 3 mg/kg at week 1, week 4, and every 2 weeks thereafter until disease progression, complete response (CR), or for a maximum of 2 years.

The primary objective was to evaluate the safety and tolerability of nivolumab. Secondary objectives included characterizing the efficacy of nivolumab and assessing PD-1 ligand loci and expression.

Adverse events (AEs) were assessed continuously during the study and for 100 days following the last treatment according to the National Cancer Institute Common Terminology Criteria for Adverse Events version 4. Patients were evaluated for efficacy at weeks 4, 8, 16, 24, and every 16 weeks thereafter.

### Study Oversight

The study protocol was approved by the institutional review board at each participating center, and the study was conducted in accordance with the Declaration of Helsinki and the International Conference on Harmonisation Guidelines for Good Clinical Practice. All patients provided written informed consent prior to study entry. Principal investigators, in collaboration with the sponsor (Bristol-Myers Squibb, Lawrenceville, N.J.), were responsible for the design and oversight of the study and for developing the protocol. The sponsor was responsible for the collection and maintenance of the data. Manuscript drafts were prepared by the authors with editorial assistance from a professional medical writer paid by the sponsor. All authors vouch for the accuracy and completeness of the data reported, adherence to the protocol, and made the decision to submit the manuscript for publication.

### Biomarker Assessment

Fluorescence in situ hybridization (FISH) was performed on cHL tissue sections to assess 9p24.1 copy number. The bacterial artificial chromosome probes (CHORI) RP11-599H2O, which maps to 9p24.1 and includes CD274 (encoding PD-L1, labeled in Spectrum Orange), and RP11-635N21, which also maps to 9p24.1 and includes PDCDJLG2 (encoding PD-L2, labeled in Spectrum Green), were co-hybridized. A control centromeric probe, Spectrum Aqua-labeled EP9 (Abbott Molecular, Inc, Abbott Park, Ill.) that maps to 9p11-q11 was hybridized following manufacturer's recommendations. Malignant HRS cells were identified by nuclear morphology, and all HRS cells were analyzed. Nuclei with a target: control probe ratio greater to or equal than three were classified as amplified; nuclei with a target:control probe ratio between one and three were identified as having relative copy gain; and nuclei with a target:control probe ratio greater than two copies of both probes were interpreted as polysomic for chromosome 9p. An automated staining system (BOND-III; Leica Biosystems, Buffalo Grove, Ill.) was employed for immunohistochemical staining, using a double-staining technique for PD-L1 (405.9A11; generated in the laboratory of G. Freeman) and PAXS (24/PAX-5; BD Biosciences, San Jose, Calif.) and for PD-L2 (366C.9E5; generated in the laboratory of G. Freeman) and p-STAT3 (D3A7; Cell Signaling Technology, Danvers, Mass.). The methods are detailed in the Supplementary Appendix.

### Statistical Analyses

All patients with relapsed/refractory cHL who received at least one dose of nivolumab were included in the safety and efficacy analyses from this ongoing study. The database lock date was Jun. 16, 2014. AEs were coded using the Medical Dictionary for Regulatory Activities version 17.0 and tabulated by system organ class and preferred term.

Efficacy assessments were evaluated by the principal investigator at each site utilizing the Revised Response Criteria for Malignant Lymphomas²². The best overall response was defined as the best response designation over the study as a whole, recorded between the date of the first dose and the last efficacy assessment prior to subsequent therapy. Objective-response rate (ORR) was defined as the proportion of the total number of patients whose best overall response was either a partial response (PR) or a CR. The duration of response was defined as the time from the date of the first documented response (CR or PR) to the date of the first documented disease progression, death, or removal from protocol to pursue stem-cell transplantation. Progression-free survival (PFS) was defined as the time from the date of the first dose of study medication to the date of first disease progression or death. The median duration of responses and PFS were estimated by Kaplan-Meier methodology. Plots of the percent changes in tumor burden over time for each patient are presented graphically.

### International Workshop to Standardize Response Criteria for Lymphoma¹

### Responses Must Last for at Least 4 Weeks Off Treatment.

Complete remission (CR): Complete disappearance of all detectable clinical and radiographic evidence of disease and disappearance of all disease-related symptoms if present before therapy and normalization of those biochemical abnormalities (for example, lactate dehydrogenase [LDH]) definitely assignable to the lymphoma. All lymph nodes must have regressed to normal size (≦1.5 cm in greatest diameter if >1.5 cm before therapy). Previously involved nodes that were 1.1 to 1.5 cm in greatest diameter must have decreased to ≦1 cm or by more than 75% in the sum of the products of the greatest diameters (SPD). The spleen, if considered to be enlarged before therapy, must have regressed in size and not be palpable on physical examination. The bone marrow must show no evidence of disease by histology. Flow cytometry, molecular or cytogenetic studies will not be used to determine response. Response must persist for 1 month. For fluorodeoxyglucose (FDG)-avid or positron-emission tomography (PET)-positive lesions prior to therapy, mass of any size is permitted if the current scan is PET negative. For variably FDG-avid or PET-negative lesions, regression to normal size on computed tomography (CT) is required.

Partial response (PR): ≧50% decreased in SPD of six largest dominant nodes or nodal masses. No increase in size of nodes, liver, or spleen and no new sites of disease. Splenic and hepatic nodules must regress by ≧50% in the SPD. Bone marrow is irrelevant for determination of a PR. No new sites of disease should be observed. For FDG-avid or PET-positive lesions prior to therapy, one or more PET positive at previously involved site is permitted. For variably FDG-avid or PET-negative lesions, regression on CT is required.

Progressive disease (PR, non-responders) requires the following: 50% increase from nadir in the SPD of any previously identified abnormal node for PRs or non-responders. Appearance of any new lesion during or at the end of therapy.

Stable disease (SD): Defined as less than a PR but not progressive disease. ALL assessment of clinical response will be made according to the non-Hodgkin lymphoma guidelines.

Relapsed disease (CR) requires the following: Appearance of any new lesion or increase by ≧50% in the size of the previously involved sites. Greater than or equal to 50% increase in greatest diameter of any previously identified node >1 cm in its shortest axis or in the SPD of more than one node.

The major criteria for judging response will include physical examination and examination of blood and bone marrow. All laboratory studies that are abnormal prior to study will be repeated to document the degree of maximal response.

### Methods for Assessment of 9p24.1 Copy Number by Fluorescence In Situ Hybridization (FISH) and Immunohistochemical (IHC) Staining

### Fluorescence In Situ Hybridization

Five micron tissue sections were mounted on standard glass slides and baked at 60° C. for at least 2 hours, then de-paraffinized and digested for 5 minutes as described previously.²

The following two bacterial artificial chromosome (BAC) probes were co-hybridized: RP11-599H20 (labeled in Spectrum Orange), which maps to 9p24.1 and includes CD274 (encoding PD-L1, hereafter PD-L1) and RP11-635N21 (labeled in Spectrum Green), which also maps to 9p24.1 and includes PDCD1LG2 (encoding PD-L2, hereafter PD-L2). Both BAC clones were obtained from CHORI. These DNA probes were direct-labeled using nick translation, precipitated using standard protocols, and hybridized with a final probe concentration of 100 ng/ul. A control centromeric probe, Spectrum Aqua-labeled CEPS (maps to 9p11-q11), was obtained from Abbott Molecular, Inc. (Abbott Park, Ill.) and was hybridized following manufacturer's recommendations.

The tissue sections and probes were co-denatured at 80° C. for 5 minutes, hybridized at least 16 hours at 37° C. in a darkened humid chamber, washed in 2×SSC at 70° C. for 10 minutes, rinsed in room temperature 2×SSC, and counterstained with DAPI (4′,6-diamidino-2-phenylindole; Abbott Molecular, Inc.). Slides were imaged using an Olympus BX51 fluorescence microscope. Individual images were captured using an Applied Imaging system running CytoVision Genus version 3.92.

Malignant Hodgkin Reed-Sternberg (RS) cells were identified by nuclear morphology and all RS cells/slide were analyzed. Nuclei with a target:control probe ratio of three or greater were classified as amplified for the target locus. Nuclei with a target:control probe ratio of between one and three were scored as having relative gain of the target locus. A target:control probe ratio of one, but with greater than two copies of each probe, was interpreted as being polysomic for the probes.

### ImmunoHistoChemical Staining

### Anti-PD-L1 and Anti-PAXS

Double staining of PD-L1 (405.9A11, from G. Freeman) and PAXS (24/PAX-5, BD Biosciences, San Jose, Calif.) was performed using an automated staining system (BOND-III, Leica Biosystems, Buffalo Grove, Ill.) following the manufacturer's protocols. Four-μm thick paraffin-embedded sections were pre-baked at 60° C. for 1 hour and subsequently loaded onto BOND-III with “Bond Universal Covertiles” (Leica Biosystems). After slides were dewaxed and rehydrated, heat-induced antigen retrieval was performed using ER2 solution (pH8) (Leica Biosystems) for 30 minutes. PD-L1 immunostaining was performed first. Primary antibody (1:100 dilution of clone 405.9A11 [final concentration: 13 μg/ml] in Ab Discovery Diluent, Ventana Medical Systems, Tucson, Ariz.) was incubated for a total of 2 hours with two separate applications, followed by 8 minutes of post-primary blocking reagent, 12 minutes of horseradish peroxidase-labeled polymer, 5 minutes of peroxidase block, and 15 minutes of DAB development. All reagents were components of the Bond Polymer Refine detection system (Leica Biosystems). PAXS (24/PAX-5) immunostaining was subsequently performed. PAXS primary antibody (1:100 dilution [final concentration: 2.50 μg/ml] in Bond Primary Antibody Diluent) was incubated for a total of 2 hours with two separate applications followed by 20 minutes of post-primary AP-blocking reagent, 15 minutes of AP-labeled polymer, and 10 minutes of Red substrate development. Slides were then counterstained with hematoxylin for 10 minutes. All reagents were components of the Bond Polymer AP Red detection system (Leica Biosystems). Slides were subsequently dehydrated and coverslipped.

### Anti-PD-L2 and Anti-Phospho STAT3

Double staining of PD-L2 (366C.9E5, from G. Freeman) and p-STAT3 (D3A7; Cell Signaling Technology, Danvers, Mass.) was performed as described above with the following modifications. PD-L2 (366C.9E5) immunostaining was performed first. Primary antibody (1:6000 dilution of clone 366C.9E5 [final concentration: 0.23 μg/ml] in Discovery Ab diluent) was incubated for a total of 2 hours with two separate applications, followed by 8 minutes of post-primary blocking reagent, 12 minutes of horseradish peroxidase-labeled polymer, 5 minutes of peroxidase block, and 15 minutes of DAB development. P-STAT3 (D3A7) immunostaining was subsequently performed. P-STAT3 primary antibody (1:125 dilution of clone D3A7 [final concentration: 0.89 μg/ml] in Bond Primary Antibody Diluent) was incubated for total of 2 hours with two separate applications. Post-primary AP blocking and labeling reagents were applied and slides were counterstained by hematoxylin as above and subsequently dehydrated and coverslipped.

### Results

### Baseline Patient Characteristics

Twenty three patients with relapsed/refractory cHL were enrolled in this study. Baseline characteristics for the patients are presented in Table 1. Median age was 35 years (20-54), and the majority of patients, 74% (n=17), had an ECOG status of 1. All patients had been extensively pretreated, with 87% having received ≧3 prior treatment regimens, 78% prior brentuximab vedotin (BV) treatment and 78% prior ASCT. Extranodal disease involving bone, lung, pelvis, peritoneum, or pleura was found in 17% of patients. With one exception, all patients had nodular sclerosis HL; the remaining patient had mixed cellularity histology. The most common first and second line chemotherapy regimens, respectively, were ABVD (doxorubicin, bleomycin, vinblastine, dacarbazine) in 20 patients (87%) and ICE (ifosfamide, carboplatin, etoposide) in 11 patients (48%).

TABLE 1
Baseline Patient Characteristics.

| Characteristic | N = 23 |
|---|---|
| Age - yr | |
| Median | 35 |
| Range | 20-54 |
| Sex - no. (%) | |
| Female | 11 (48) |
| Male | 12 (52) |
| Race - no. (%) | |
| White | 20 (87) |
| Black/African American | 2 (9) |
| Other | 1 (4) |
| ECOG performance status* - no. (%) | |
| 0 - Asymptomatic | 5 (22) |
| 1 - Ambulatory but restricted in strenuous activity | 17 (74) |
| Histology - no. (%) | |
| Nodular sclerosis | 22 (96) |
| Mixed cellularity | 1 (4) |
| Prior systemic therapies - no. (%) | |
| 2 to 3 | 8 (35) |
| 4 to 5 | 7 (30) |
| 6 or more | 8 (35) |
| Prior brentuximab vedotin - no. (%) | |
| Yes | 18 (78) |
| No | 5 (22) |
| Prior ASCT - no. (%) | |
| Yes | 18 (78) |
| No | 5 (22) |
| Prior radiation therapy - no. (%) | |
| Yes | 19 (83) |
| No | 4 (17) |
| Extranodal involvement† - no. (%) | |
| Yes | 4 (17) |
| No | 19 (83) |

*Eastern Cooperative Oncology Group (ECOG) performance status is missing data for 1 patient.
†Sites of extranodal disease were bone, lung, pelvis, peritoneum, and pleura.
ASCT, autologous stem-cell transplantation.

### Safety

Among the 23 patients with cHL in this study, AEs of any grade were reported in 22 patients (96%). Grade 3 or 4 AEs occurred in 12 patients (52%). Drug-related AEs that occurred in ≧5% of patients are shown in Table 2. Overall, drug-related AEs were reported in 18 patients (78%). The most common were rash (22%) and decreased platelet count (17%). Drug-related grade 3 or 4 AEs were reported in 5 patients (22%) and included myelodysplastic syndrome (MDS), pancreatitis, pneumonitis, stomatitis, colitis, gastrointestinal inflammation, low platelets, increased lipase, decreased lymphocytes, and decreased leukocytes. Three patients experienced one serious drug-related AE each (grade 3 pancreatitis, grade 3 MDS, and grade 2 lymph node pain; Table 2). The patient who developed MDS had received multiple courses of prior therapy, including ABVD and two courses of ICE (ifosfamide, carboplatin, etoposide). There were no treatment-related deaths.

Twelve patients (57%) discontinued treatment: 2 patients (9%) for toxicity (MDS and thrombocytopenia in 1 patient; pancreatitis in 1 patient), 4 patients (22%) who progressed during treatment, and 6 patients (26%) with a best overall response of CR or PR who elected to proceed to allogeneic (n=5) or autologous (n=1) stem-cell transplantation. Eleven patients (43%) continue on study as of Jun. 16, 2014.

Patients received a median of 16 doses of nivolumab (range, 6-37) over a median treatment duration of 36 weeks (range, 13-77). The median dose intensity per patient was 1.4 mg/kg/week, with 15 patients (65%) receiving ≧90% of the intended dose. Nine patients (39%) had at least one dose delay. Two patients (9%) had infusion interruptions due to grade 1 hypersensitivity reactions.

TABLE 2
Drug-Related Adverse Events.

| Event - no. (%) | Any Grade | Grade 3 or 4 |
|---|---|---|
| Total | 18 (78) | 5 (22) |
| Drug-related adverse events reported in ≧5% | | |
| of patients | | |
| Rash | 5 (22) | 0 |
| Decreased platelet count | 4 (17) | 0 |
| Fatigue | 3 (13) | 0 |
| Pyrexia | 3 (13) | 0 |
| Diarrhea | 3 (13) | 0 |
| Nausea | 3 (13) | 0 |
| Pruritus | 3 (13) | 0 |
| Cough | 2 (9) | 0 |
| Hypothyroidism | 2 (9) | 0 |
| Decreased lymphocyte count | 2 (9) | 1 (4) |
| Hypophosphatemia | 2 (9) | 0 |
| Hypercalcemia | 2 (9) | 0 |
| Increased lipase | 2 (9) | 1 (4) |
| Stomatitis | 2 (9) | 1 (4) |
| Drug-related serious adverse events | | |
| Myelodysplastic syndrome | 1 (4) | 1 (4) |
| Lymph node pain | 1 (4) | 0 |
| Pancreatitis | 1 (4) | 1 (4) |

### Clinical Activity

In these heavily pretreated patients, the ORR was 87% (95% confidence interval [CI], 66 to 97), with CR occurring in 4 patients (17%), PR in 16 patients (70%), and stable disease (SD) in 3 patients (13%) (Table 3). Results are also summarized by three subgroups: patients who failed a prior ASCT and prior BV; patients who failed BV but did not have a prior ASCT (ASCT-naïve); and patients who did not receive BV (BV-naïve) (Table 3). Among 15 patients who had disease recurrence following ASCT and BV, the ORR was 87% (95% CI, 60 to 98), with 1 patient (7%) achieving CR, 12 (80%) PR, and 2 (13%) SD. For the group of 3 patients who were ASCT-naïve before BV, the ORR was 100% (95% CI, 29 to 100), with 3 patients achieving PR. Among the 5 BV-naïve patients, the ORR was 80% (95% CI, 28 to 99), with 3 patients (60%) achieving CR, 1 (20%) PR, and 1 (20%) SD.

The median duration of response was 52 weeks (95% CI, 18 to 52); however, the interpretation of this finding is limited by the high number of ongoing responders (n=11; 55%). Of the 20 patients that had a response (CR or PR), 12 (60%) patients achieved their first response by 8 weeks. After achieving a best overall response of CR, PR, or SD, the PFS rate at 24 weeks was 86% (95% CI, 62 to 95). Median overall survival has not been reached (range, 21+ to 75+ weeks).

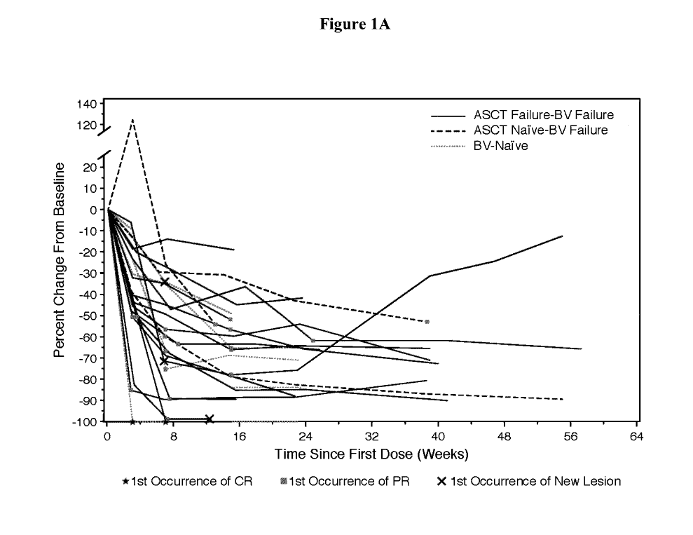

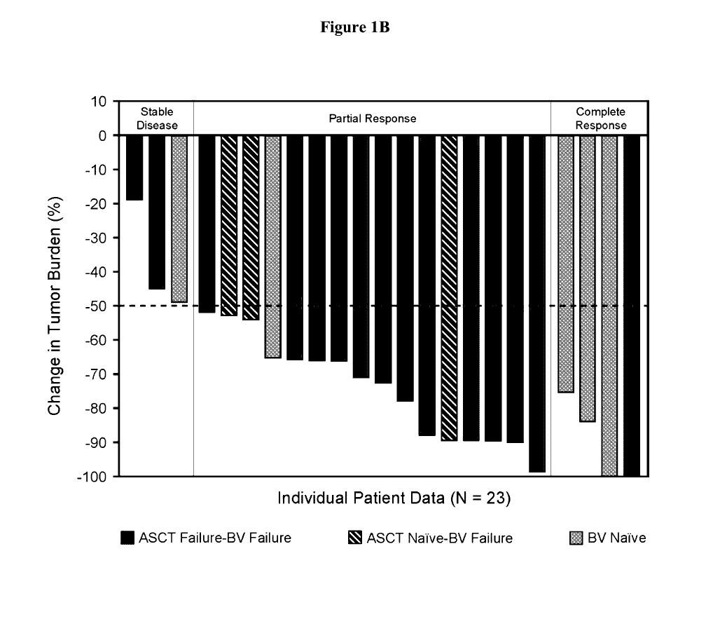

FIG. 1A depicts change in target tumor burden from baseline over time for each patient. FIG. 1B shows the maximum reduction in tumor burden from baseline for each patient.

TABLE 3
Clinical Activity in Nivolumab-Treated Patients.

| | | ASCT | ASCT- | |
|---|---|---|---|---|
| | | Failure/BV | Naïve/BV | BV- |
| | Total | Failure | Failure | Naïve* |
| | (N = 23) | (n = 15) | (n = 3) | (n = 5) |
| Best overall response - no. (%) | | | | |
| CR | 4 (17) | 1 (7) | 0 | 3 (60) |
| PR | 16 (70) | 12 (80) | 3 (100) | 1 (20) |
| SD | 3 (13) | 2 (13) | 0 | 1 (20) |
| PD | 0 | 0 | 0 | 0 |
| Objective-response rate - no. (%) | 20 (87) | 13 (87) | 3 (100) | 4 (80) |
| 95% CI | (66-97) | (60-98) | (29-100) | (28-99) |
| PFS at 24 weeks - | 86 (62-95) | 85 (52-96) | NC§ | 80 (20-97) |
| % (95% CI)‡ | | | | |
| Median overall survival - | NR | NR | NR | NR |
| weeks | | | | |
| Range | 21+, 75+ | 21+, 75+ | 32+, 55+ | 30+, 50+ |

*Brentuximab vedotin (BV)-naive group consists of patients with and without prior autologous stem-cell transplantation (ASCT; n = 2 and n = 3, respectively).
‡Point estimates are derived from Kaplan-Meier analyses; the 95% confidence intervals (CIs) are derived from Greenwood's formula.
§Estimator was not calculated when the percentage of censoring was above 25%.
+censored
NC, not calculated;
NR, not reached;
PD, progressive disease;
PFS, progression-free survival;
SD, stable disease.

### Analysis of Pd-1 Ligand Loci and Expression

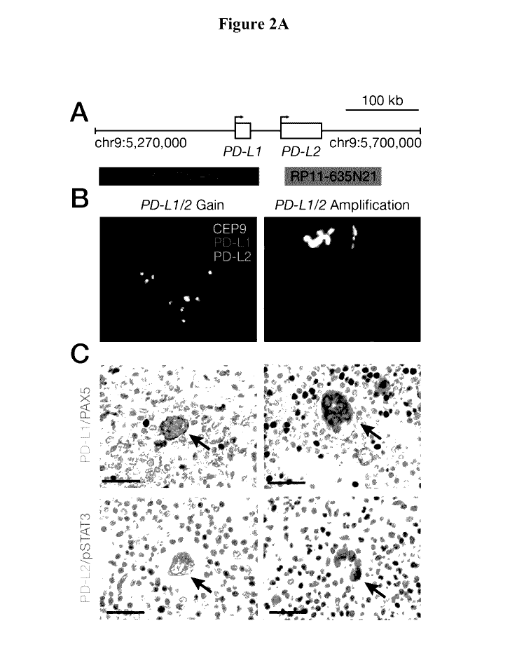

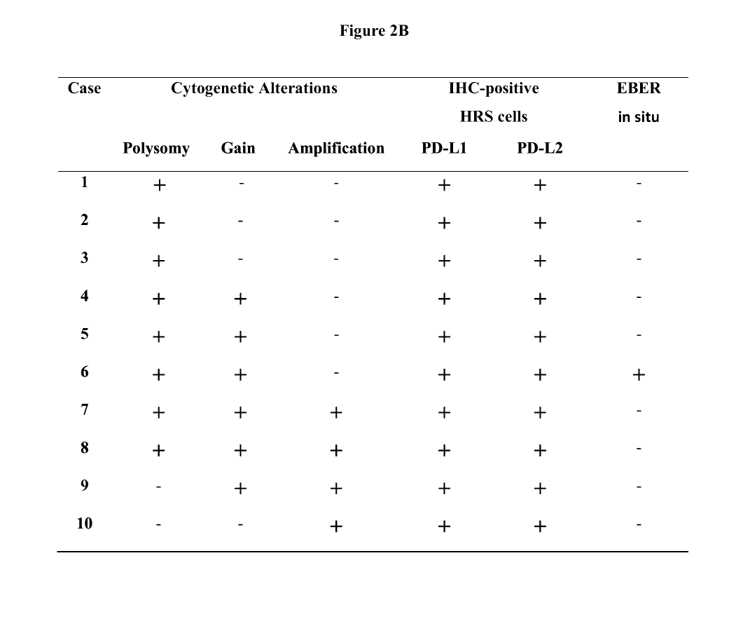

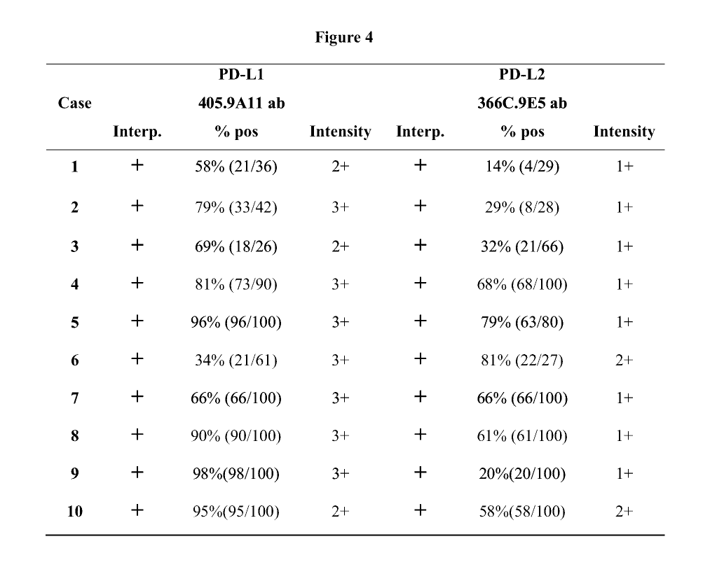

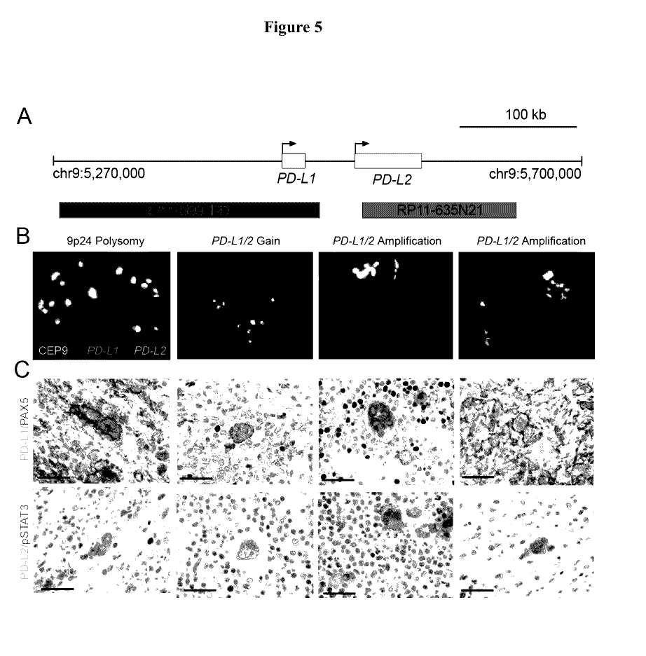

In a subset of study patients (n=10/23), PD-L1 and PD-L2 copy numbers in HRS cells were assessed using fixed tumor biopsy specimens and a three-probe FISH assay (CD274, PD-L1, red; PDCDJLG2, PD-L2, green; and control centromeric probe, aqua; FIG. 2A). In all tumors analyzed by FISH, HRS cells had three to 15 copies of PD-L1 and PD-L2 in patterns including amplification, relative copy gain, or polysomy of chromosome 9p (FIG. 2B). In all cases, HRS cells, which were identified by their characteristic morphology and PAXS staining, expressed the PD-L1 and PD-L2 proteins (FIGS. 2A and 2B, and FIGS. 4 and 5). HRS cells were also largely positive for pSTAT3, indicative of active JAK-STAT signaling (FIG. 2A, and FIG. 4). Therefore, all evaluable patients with cHL in this study had genetic alterations of the PD-1 ligand loci and associated protein expression.

What is claimed is:

1. A method for treating a subject afflicted with a Hodgkin's lymphoma comprising administering to the subject a therapeutically effective amount of an antibody or an antigen-binding portion thereof that binds specifically to a Programmed Death-1 receptor (PD-1).

2. The method of claim 1, wherein the Hodgkin's lymphoma expresses PD-L1 and/or PD-L2.

3. The method of claim 2, wherein the PD-L1 and/or PD-L2 expression is at least about 1%.

4. A method for identifying a subject afflicted with a tumor derived from a Hodgkin's lymphoma who is suitable for an anti-PD1 antibody therapy comprising measuring PD-L1 and/or PD-L2 expression on the tumor, wherein the tumor has a PD-L1 and/or PD-L2 expression of at least about 1% and wherein the subject is administered a therapeutically effective amount of an anti-PD-1 antibody or antigen-binding portion thereof.

5. The method of claim 4, wherein the PD-L1 and/or PD-L2 expression of the tumor is at least about 5%.

6. The method of any claim 4, wherein the PD-L1 and/or PD-L1 expression of the tumor is at least about 10%.

7. The method of claim 4, wherein the anti-PD-1 antibody or antigen-binding portion thereof cross-competes with nivolumab for binding to human PD-1.

8. The method of claim 7, wherein the anti-PD-1 antibody or antigen-binding portion thereof is a chimeric, humanized or human monoclonal antibody or a portion thereof.

9. The method of claim 8, wherein the anti-PD-1 antibody or antigen-binding portion thereof comprises a heavy chain constant region which is of a human IgG1 or IgG4 isotype.

10. The method of claim 9, wherein the anti-PD-1 antibody is nivolumab.

11. The method of claim 9, wherein the anti-PD-1 antibody is pembrolizumab.

12. The method of claim 9, which further comprises administering one or more additional anti-cancer agent.

13. The method of claim 12, wherein the anti-cancer agent is selected from the group consisting of an antibody or antigen-binding portion thereof that binds specifically to a CTLA-4 and inhibits CTLA-4 activity, a chemotherapy, a platinum-based doublet chemotherapy or a tyrosine kinase inhibitor.

14. The method of claim 12, wherein the anti-cancer agent is an antibody or antigen-binding portion thereof that binds specifically to a CTLA-4 and inhibits CTLA-4 activity.

15. The method of claim 9, wherein the PD-L1 and/or PD-L2 expression is measured by automated immunohistochemistry (IHC) or fluorescent in situ hybridization.

16. A kit for treating a subject afflicted with a tumor derived from a Hodgkin's lymphoma, the kit comprising:
(a) an anti-PD-1 antibody or antigen-binding portion thereof;
(b) instructions for determining the PD-L1 and/or PD-L2 expression of the tumor and, if the tumor expresses PD-L1 or PD-L2, administering the anti-PD-1 antibody or antigen-binding portion thereof to the subject in the methods of claim 15.

17. The kit of claim 16, further comprising an agent to determine the PD-L1 and/or PD-L2 expression of the tumor.

18. The kit of claim 17, wherein the PD-L1 and/or PD-L2 expression is measured by an anti-PD-L1 and/or PD-L2 antibody or antigen-binding portion thereof.
